(19) United States
(12) Patent Application Publication
(10) Pub. No.: US 2022/0251235 A1
(43) Pub. Date: Aug. 11, 2022

(54) MAGEA10 SPECIFIC T CELL RECEPTORS AND THEIR USE

(71) Applicant: MEDIGENE IMMUNOTHERAPIES GMBH, Planegg-Martinsried (DE)

(72) Inventors: Daniel SOMMERMEYER, Muenchen (DE); Christian ELLINGER, Muenchen (DE); Aline BRACHER, Muenchen (DE); Christiane MUMMERT, Planegg (DE); Laura PRASSMAYER, Rosenheim (DE); Raphaela PUTZHAMMER, Muenchen (DE); Alexandra SEMMELMANN, Alling (DE)

(21) Appl. No.: 17/625,380

(22) PCT Filed: Jul. 8, 2020
(86) PCT No.: PCT/EP2020/069246

§ 371 Date: Jan. 7, 2022

(30) Foreign Application Priority Data
Jul. 9, 2019 (EP) 19185192.2

(51) Int. Cl. C07K 16/30; C07K 14/47; C07K 14/725; A61P 35/00

(52) U.S. Cl. CPC C07K 16/30; C07K 14/4748; A61K 2039/505; A61P 35/00; C07K 14/7051

(57) ABSTRACT

The present invention relates to an isolated T cell receptor (TCR) specific for a MAGEA10-derived peptide and to a polypeptide comprising a functional portion of the TCR. Further implicated are a multivalent TCR complex, a nucleic acid encoding a TCR, a cell expressing the TCR and a pharmaceutical composition comprising the TCR. The invention also refers to the TCR for use as a medicament, in particular to the TCR for use in the treatment of cancer.

### FIELD OF THE INVENTION

The present invention relates to an isolated T cell receptor (TCR) specific for a MAGEA10-derived peptide and to a polypeptide comprising a functional portion of the TCR. Further implicated are a multivalent TCR complex, a nucleic acid encoding a TCR, a cell expressing the TCR and a pharmaceutical composition comprising the TCR. The invention also refers to the TCR for use as a medicament, in particular to the TCR for use in the treatment of cancer.

### BACKGROUND OF THE INVENTION

MAGEA10, also called Melanoma-Associated Antigen 10, is a member of the MAGEA gene family coding for a number of proteins having high homology with each other. The MAGEA antigens belong to the family of cancer/testis antigens (CTA) and were the first human tumor-associated antigens identified at the molecular level (Science 1991. 254: 1643-1647/republished in J. Immunol. 2007; 178:2617-2621). The MAGEA gene family includes 12 highly homologous genes located on chromosome Xq28. Their expression is consistently detected in cancers of different histological origin, such as non-small cell lung cancer, bladder cancer, esophageal, head and neck cancer and sarcoma, as well as myeloma, certain types of breast cancer and also in germinal cells (Front Med (Lausanne). 2017; 4: 18.). MAGEA10 is a cytosolic/cytoplasmic protein and peptides derived from the MAGEA10 protein are presented in an MHC-class I background, i.e. on HLA molecules. More specifically, MAGEA10 derived epitopes are presented on HLA-A2 molecules, indicating their suitability as targets for cancer immunotherapy. A multitude of clinical trials in the field of immunotherapy have already been conducted targeting MAGEA proteins (clinicaltrials.gov). MAGEA10 is a promising target for T cell-mediated immunotherapy. The principle of cancer immunotherapy using adoptive cell transfer (ACT) enables a highly tumor specific cancer treatment utilizing a patient's own immune system. ACT uses ex vivo expanded autologous (patient-derived) T cells genetically engineered to express T cell receptors (TCR) with a specificity for a defined epitope derived from an intracellular protein like MAGEA10. Thus, there is still a need for highly efficient TCRs which target only the tumor specific/restricted antigen MAGEA10 and therefore bear an exceptional potential for cancer immunotherapy. Thus, there is a need for such specific TCRs targeting MAGEA10.

### OBJECTIVES AND SUMMARY OF THE INVENTION

One objective of the present invention is the provision of a T cell receptor (TCR) which is specific for MAGEA10.

In a specific embodiment the isolated TCR comprises a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7.

The TCR specifically recognizes the amino acid sequence SEQ ID NO: 1 or a fragment thereof. In specific embodiments, the TCR specifically recognizes the HLA-A2 bound form of the amino acid sequence of SEQ ID NO: 1. More specifically, the TCR specifically recognizes the amino acid sequence of SEQ ID NO: 1, which is presented by a molecule encoded by a gene selected from the group consisting of HLA-A*02:01 and HLA-A*02:04, more preferably the TCR specifically recognizes the amino acid sequence of SEQ ID NO: 1, which is presented by the HLA-A*02:01 encoded molecule.

The fragment may be a sequence of the antigen that is specific for this antigen, i.e. does not occur in another protein or peptide of a mammal, especially of a human. The fragment may be shorter than the sequence of the antigen, such as at least 5%, at least 10%, at least 30%, at least 50%, at least 70%, at least 90% shorter than the antigen. The fragment may have a length of at least 9, at least 10, at least 11, at least 12, at least 13, at least 14, at least 15 or more amino acids.

Preferably the TCR does not have cross-reactivity to other MAGEA family members.

Another objective of the present invention is the provision of T cells expressing a functional TCR being specific for MAGEA10.

The TCR according to the invention is isolated and/or purified and may be soluble or membrane bound.

In some embodiments, the amino acid sequence of the TCR may comprise one or more phenotypically silent substitutions. In addition, the TCR of the invention can be labelled with a detectable label. Additionally, or alternatively, the amino acid sequence may be modified to comprise a therapeutic agent or pharmacokinetic modifying moiety. The therapeutic agent may be selected from the group consisting of an immune effector molecule, a cytotoxic agent and a radionuclide. The immune effector molecule may for example be a cytokine. The pharmacokinetic modifying moiety may be at least one polyethylene glycol repeating unit, at least one glycol group, at least one sialyl group or a combination thereof.

The TCR, in particular a soluble form of the TCR, according to the invention can be modified by attaching additional functional moieties, e.g. for reducing immunogenicity, increasing hydrodynamic size (size in solution) solubility and/or stability (e.g. by enhanced protection to proteolytic degradation) and/or extending serum half-life. Other useful functional moieties and modifications include “suicide” or “safety switches” that can be used to shut off or turn on effector host cells carrying an inventive TCR in a patient's body or to shut off or turn on the transgenic TCR itself. TCRs with an altered glycosylation pattern are also envisaged herein.

It is also conceivable to add a drug or a therapeutic entity, such as a small molecule compound to the TCR, in particular to a soluble form of the inventive TCR.

The TCR, in particular a soluble form of the inventive TCR, can additionally be modified to introduce additional domains which aid in identification, tracking, purification and/or isolation of the respective molecule (tags).

In some embodiments, the TCR is of the single chain type, wherein the TCR α chain and the TCR β chain are linked by a linker sequence.

Another aspect of the invention refers to a polypeptide comprising a functional portion of the TCR as described herein, wherein the functional portion comprises one of the amino acid sequences of SEQ ID NOs: 4 and 7, preferably wherein the functional portion comprises the amino acid sequences of SEQ ID NOs: 2, 3, 4 and 5, 6, 7.

In specific embodiments, the functional portion comprises the TCR α variable chain and/or the TCR β variable chain.

Specific embodiments refer to a multivalent TCR complex comprising at least two TCRs as described herein. In a more specific embodiment, at least one of said TCRs is associated with a therapeutic agent.

Some embodiments refer to the inventive TCR expressed on an effector cell, especially on an immune effector cell as a functional polypeptide or functional multivalent polypeptide, wherein IFN-γ secretion is induced in the aforementioned effector cell expressing the TCR upon binding to an HLA-A2 bound form of the amino acid sequence SEQ ID NO: 1.

Another aspect of the invention refers to a nucleic acid encoding a TCR as described herein or encoding the polypeptide as described above.

A further aspect of the invention refers to a plasmid or vector comprising the nucleic acid of the present application as described above. Preferably, the vector is an expression vector or a vector suitable for the transduction or transfection of cells, especially eukaryotic cells. The vector may be for example a retroviral vector, for example a gamma-retroviral or lentiviral vector.

Another aspect of the invention refers to a cell expressing the TCR as described herein. The cell may be an isolated primary cell or non-naturally occurring.

Another aspect of the invention refers to a cell comprising the nucleic acid as described above or the plasmid or vector as described above. More specifically, the cell may comprise:

a) an expression vector which comprises at least one nucleic acid as described above, or

b) a first expression vector which comprises a nucleic acid encoding the alpha chain of the TCR as described herein, and a second expression vector which comprises a nucleic acid encoding the beta chain of a TCR as described herein.

The cell may be a peripheral blood lymphocyte (PBL) or a peripheral blood mononuclear cell (PBMC). Typically, the cell is an immune effector cell, especially a T cell. Other suitable cell types include gamma-delta T cells and NK-like T cells and NK cells, either modified or unmodified.

Another aspect refers to an antibody or antigen binding fragment thereof specifically binding to a portion of the TCR as described herein which mediates specificity for MAGEA10. In a specific embodiment, the portion of the TCR that mediates the MAGEA10 specificity comprises the CDR3 of the alpha chain of SEQ ID NO: 4 and/or the CDR3 of the beta chain of SEQ ID NO: 7. In some embodiments the portion of the TCR that mediates the MAGEA10 specificity comprises the amino acid sequences of SEQ ID NOs: 2, 3, 4 and 5, 6, 7.

Another aspect of the invention refers to a pharmaceutical composition comprising the TCR as described herein, the polypeptide as described herein, the multivalent TCR complex as described herein, the nucleic acid as described herein, the vector as described herein, the cell as described herein, or the antibody as described herein.

Typically, the pharmaceutical composition comprises at least one pharmaceutically acceptable carrier.

Another aspect of the invention refers to the TCR as described herein, the polypeptide as described herein, the multivalent TCR complex as described herein, the nucleic acid as described herein, the vector as described herein, the cell as described herein, or the antibody as described herein for use as a medicament, in particular for use in the treatment of cancer. The cancer may be a hematological cancer or a solid tumor. The cancer may be selected from the group consisting of prostate cancer, uterine cancer, thyroid cancer, testicular cancer, renal cancer, pancreatic cancer, ovarian cancer, esophageal cancer, non-small-cell lung cancer, lung adenocarcinoma, squamous cell carcinoma, non-Hodgkin's lymphoma, multiple myeloma, melanoma, hepatocellular carcinoma, head and neck cancer, gastric cancer, endometrial cancer, cervical cancer, colorectal cancer, stomach adenocarcinoma, cholangiocarcinoma, breast cancer, bladder cancer, myeloid leukemia and acute lymphoblastic leukemia, sarcoma or osteosarcoma.

### FIGURE LEGENDS

FIG. 1 shows the MAGE-A10^{SLL}-MHC-multimer binding of CD8⁺ T cells transduced with a MAGE-A10-reactive TCR. CD8 T cells were isolated from PBMCs of a healthy donor and transduced with the MAGE-A10^{SLL}-TCR and a negative control TCR that did not recognize MAGE-A10^{SLL}. Transduced CD8 T cells were enriched by FACS using the murine constant beta region as a marker for transduction. After expansion of these cells, they were stained with a MAGE-A10^{SLL}-MHC-multimer (SLL-tetramer) and antibodies against CD8 and the murine constant beta region (mCß) and analyzed by flow cytometry.

FIG. 2 shows that MAGE-A10^{SLL}-TCR-transgenic T cells recognize MAGE-A10^{SLL}-peptide presented on HLA-A2. Transgenic T cells were co-cultured with T2 cells externally loaded with MAGE-A10^{SLL}-peptide. T2 cells loaded with a control peptide were used as negative control. Recognition of target cells was analyzed by measuring the IFN-γ concentration in co-culture supernatants by a standard ELISA. FIG. 2 also shows that a control TCR does not recognize the MAGE-A10^{SLL} epitope.

FIGS. 3A and 3B show the ability of MAGE-A10^{SLL}-TCR-transgenic T cells to recognize MAGE-A10^{SLL} presented on tumor cell lines. Transgenic T cells were co-cultured with different MAGE-A10^{SLL}-positive tumor cell lines. As negative control two cell lines (Mel624.38 and 647V) not expressing MAGE-A10 were used. (A) Cytokine release was analyzed by measuring the IFN-γ concentration in co-culture supernatants by a standard ELISA. (B) Cytotoxicity against tumor cell lines stably transduced with a fluorescence marker was measured with an IncuCyte.

FIG. 4 shows the recognition of MAGE-A family members by the MAGE-A10^{SLL}-TCR. Transgenic T cells were co-cultured with K562 cells expressing different MAGE-A family members. Cytokine release was analyzed by measuring the IFN-γ concentration in co-culture supernatants by a standard ELISA.

FIGS. 5A and 5B show the HLA-fine-typing for the MAGE-A10^{SLL}-TCR and the analysis of potential alloreactivity of the MAGE-A10^{SLL}-TCR. A library of (A) MAGE-A10^{SLL} peptide loaded or (B) unloaded LCL expressing different HLA-A types were co-cultured, either with MAGE-A10^{SLL}-TCR-transgenic T cells or a negative control TCR. As positive control T2 cells were externally loaded with MAGE-A10^{SLL}-peptide. Cytokine release was analyzed by measuring the IFN-γ concentration in co-culture supernatants by a standard ELISA.

FIG. 6 shows the analysis of recognition of normal cells by the MAGE-A10^{SLL}-TCR. Transgenic T cells were co-cultured with normal cells and with normal cells externally loaded with MAGE-A10^{SLL}-peptide. Cytokine release was analyzed by measuring the IFN-γ concentration in co-culture supernatants by a standard ELISA.

### DETAILED DESCRIPTION OF THE INVENTION

Before the invention is described in detail with respect to some of its preferred embodiments, the following general definitions are provided.

The present invention as illustratively described in the following may be suitably practiced in the absence of any element or elements, limitation or limitations, not specifically disclosed herein.

The present invention will be described with respect to particular embodiments and with reference to certain figures but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. an antibody is defined to be obtainable from a specific source, this is also to be understood to disclose an antibody which is obtained from this source.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±10%, and preferably of ±5%.

Technical terms are used by their common sense or meaning to the person skilled in the art. If a specific meaning is conveyed to certain terms, definitions of terms will be given in the following in the context of which the terms are used.

### TCR Background

A TCR is composed of two different and separate protein chains, namely the TCR alpha (α) and the TCR beta (β) chain. The TCR α chain comprises variable (V), joining (J) and constant (C) regions. The TCR β chain comprises variable (V), diversity (D), joining (J) and constant (C) regions. The rearranged V(D)J regions of both the TCR α and the TCR β chain contain hypervariable regions (CDR, complementarity determining regions), among which the CDR3 region determines the specific epitope recognition. At the C-terminal region both TCR α chain and TCR β chain contain a hydrophobic transmembrane domain and end in a short cytoplasmic tail.

Typically, the TCR is a heterodimer of one α chain and one β chain. This heterodimer can bind to MHC molecules presenting a peptide.

The term “variable TCR α region” or “TCR α variable chain” or “variable domain” in the context of the invention refers to the variable region of a TCR α chain. The term “variable TCR β region” or “TCR β variable chain” in the context of the invention refers to the variable region of a TCR β chain.

The TCR loci and genes are named using the International Immunogenetics (IMGT) TCR nomenclature (IMGT Database, www.IMGT.org; Giudicelli, V., et al. IMGT/LIGM-DB, the IMGT® comprehensive database of immunoglobulin and T cell receptor nucleotide sequences, Nucl. Acids Res., 34, D781-D784 (2006). PMID: 16381979; T cell Receptor Factsbook, LeFranc and LeFranc, Academic Press ISBN 0-12-441352-8).

### Target

The target for the herein described TCR is MAGEA10 (NCBI Reference Sequence: NM_001011543.2)-derived peptide SLL (SEQ ID NO: 1)

### TCR Specific Sequence

Some embodiments relate to an isolated TCR comprising a TCR α chain and a TCR β chain, wherein
the TCR α chain comprises a complementarity-determining region 3 (CDR3) having the sequence of SEQ ID NO: 4,
the TCR β chain comprises a CDR3 having the amino acid sequence of SEQ ID NO: 7.

Specific embodiments refer to an isolated TCR comprising:
a TCR α chain comprising a CDR1 having the amino acid sequence of SEQ ID NO: 2, a CDR 2 having the amino acid sequence of SEQ ID NO: 3 and a CDR 3 having the sequence of SEQ ID NO: 4.
a TCR β chain comprising a CDR1 having the amino acid sequence of SEQ ID NO: 5, a CDR 2 having the amino acid sequence of SEQ ID NO: 6 and a CDR 3 having the sequence of SEQ ID NO: 7.

In some embodiments, the TCR comprises a variable TCR α region having an amino acid sequence which is at least 80%, at least 85%, at least 90%, at least 95%, at least 96%, at least 97%, at least 98%, at least 99% identical to SEQ ID NO: 8 and a variable TCR β region having an amino acid sequence which is at least 80%, at least 85%, at least 90%, at least 95%, at least 96%, at least 97%, at least 98%, at least 99% identical to SEQ ID NO: 9.

A preferred embodiment relates to a TCR comprising a variable TCR α region having the amino acid sequence of SEQ ID NO: 8 and a variable TCR β region having the amino acid sequence of SEQ ID NO: 9.

The inventive TCR used in the examples, comprises a TCR α chain comprising a complementarity-determining region 3 (CDR3) having the sequence of SEQ ID NO: 4 and a TCR β chain comprising a CDR3 having the amino acid sequence of SEQ ID NO: 7. In particular, the inventive TCR comprises a variable TCR α region having the amino acid sequence of SEQ ID NO: 8 and a variable TCR β region having the amino acid sequence of SEQ ID NO: 9.

As can be seen from the examples the TCRs according to the invention are specific for MAGEA10 and exhibit only very low cross-reactivity to other epitopes or antigens.

Other embodiments relate to an isolated TCR comprising a TCR α chain having an amino acid sequence which is at least 80%, at least 85%, at least 90%, at least 95%, at least 96%, at least 97%, at least 98%, at least 99% identical to SEQ ID NO: 10 and a TCR β chain having an amino acid sequence which is at least 80%, at least 85%, at least 90%, at least 95%, at least 96%, at least 97%, at least 98%, at least 99% identical to SEQ ID NO: 11.

Specific embodiments refer to a TCR comprising a TCR α chain having the amino acid sequence of SEQ ID NO: 10 and a TCR β chain having the amino acid sequence of SEQ ID NO: 11. Thus, the TCR described herein that is specific for the complex of HLA-A*02:01 with the MAGEA10 peptide of SEQ ID NO: 1 comprises a Vα chain encoded by the TRAV29/DV5 gene and a Vβ gene encoded by the TRBV24-1 gene.

Other embodiments refer to an isolated TCR comprising a TCR α chain and a TCR β chain, wherein
the variable TCR α region has an amino acid sequence which is at least 80%, at least 85%, at least 90%, at least 95%, at least 96%, at least 97%, at least 98%, at least 99% identical to SEQ ID NO: 8 and comprises a CDR3 region set out in SEQ ID NO: 4;
the variable TCR β region has an amino acid sequence which is at least 80%, at least 85%, at least 90%, at least 95%, at least 96%, at least 97%, at least 98%, at least 99% identical to SEQ ID NO: 9 and comprises a CDR3 region set out in SEQ ID NO: 7.

The determination of percent identity between multiple sequences is preferably accomplished using the AlignX application of the Vector NTI Advance™ 10 program (Invitrogen Corporation, Carlsbad Calif., USA). This program uses a modified Clustal W algorithm (Thompson et al., 1994. Nucl Acids Res. 22: pp. 4673-4680; Invitrogen Corporation; Vector NTI Advance™ 10 DNA and protein sequence analysis software. User's Manual, 2004, pp. 389-662). The determination of percent identity is performed with the standard parameters of the AlignX application.

The TCR according to the invention is isolated or purified. “Isolated” in the context of the invention means that the TCR is not present in the context in which it originally occurred in nature. “Purified” in the context of the invention means e.g. that the TCR is free or substantially free of other proteins and non-protein parts of the cell it originates from.

In some embodiments, the amino acid sequence of the TCR may comprise one or more phenotypically silent substitutions.

“Phenotypically silent substitutions” are also named “conservative amino acid substitutions”. The concept of “conservative amino acid substitutions” is understood by the skilled artisan, and preferably means that codons encoding positively-charged residues (H, K, and R) are substituted with codons encoding positively-charged residues, codons encoding negatively-charged residues (D and E) are substituted with codons encoding negatively-charged residues, codons encoding neutral polar residues (C, G, N, Q, S, T, and Y) are substituted with codons encoding neutral polar residues, and codons encoding neutral non-polar residues (A, F, I, L, M, P, V, and W) are substituted with codons encoding neutral non-polar residues. These variations can spontaneously occur, be introduced by random mutagenesis, or can be introduced by directed mutagenesis. Those changes can be made without destroying the essential characteristics of these polypeptides. The ordinarily skilled artisan can readily and routinely screen variant amino acids and/or the nucleic acids encoding them to determine if these variations substantially reduce or destroy the ligand binding capacity by methods known in the art.

The skilled person understands that also the nucleic acid encoding the TCR may be modified. Useful modifications in the overall nucleic acid sequence include codon optimization of the sequence. Alterations may be made which lead to conservative substitutions within the expressed amino acid sequence. These variations can be made in complementarity determining and non-complementarity determining regions of the amino acid sequence of the TCR chain that do not affect function. Usually, additions and deletions should not be performed in the CDR3 region.

According to some embodiments of the invention the amino acid sequence of the TCR is modified to comprise a detectable label, a therapeutic agent or pharmacokinetic modifying moiety.

Non-limiting examples for detectable labels are radiolabels, fluorescent labels, nucleic acid probes, enzymes and contrast reagents. Therapeutic agents which may be associated with the TCRs include radioactive compounds, immune-modulators, enzymes or chemotherapeutic agents. The therapeutic agents could be enclosed by a liposome linked to TCR so that the compound can be released slowly at the target site. This will avoid damage during the transport in the body and ensure that the therapeutic agent, e.g. toxin, has maximum effect after binding of the TCR to the relevant antigen presenting cells. Other examples for therapeutic agents are:

peptide cytotoxins, i.e. proteins or peptides with the ability to kill mammalian cells, such as ricin, diphtheria toxin, pseudomonas bacterial exotoxin A, DNase and RNase. Small molecule cytotoxic agents, i.e. compounds with the ability to kill mammalian cells having a molecular weight of less than 700 Daltons. Such compounds could contain toxic metals capable of having a cytotoxic effect. Furthermore, it is to be understood that these small molecule cytotoxic agents also include pro-drugs, i.e. compounds that decay or are converted under physiological conditions to release cytotoxic agents. Such agents may for example include docetaxel, gemcitabine, cisplatin, maytansine derivatives, rachelmycin, calicheamicin, etoposide, ifosfamide, irinotecan, porfimer sodium photofrin II, temozolomide, topotecan, trimetrexate glucoronate, mitoxantrone, auristatin E, vincristine and doxorubicin; radionuclides, such as, iodine 131, rhenium 186, indium 111, yttrium 90. bismuth 210 and 213, actinium 225 and astatine 213. The association of the radionuclides with the TCRs or derivatives thereof may for example be carried out by chelating agents; immune-stimulators, also known as immunostimulants, i.e. immune effector molecules which stimulate immune response. Exemplary immune-stimulators are cytokines such as IL-2 and IFN-γ, antibodies or fragments thereof, including anti-T cell or NK cell determinant antibodies (e.g anti-CD3, anti-CD28 or anti-CD16); alternative protein scaffolds with antibody like binding characteristics; Superantigens, i.e. antigens that cause non-specific activation of T-cells resulting in polyclonal T cell activation and massive cytokine release, and mutants thereof; chemokines such as IL-8, platelet factor 4, melanoma growth stimulatory protein, etc. complement activators; xenogeneic protein domains, allogeneic protein domains, viral/bacterial protein domains, viral/bacterial peptides.

The antigen receptor molecules (T cell receptor molecules) on human T lymphocytes are non-covalently associated with the CD3 (T3) molecular complex on the cell surface. Perturbation of this complex with anti-CD3 monoclonal antibodies induces T cell activation. Thus, some embodiments refer to a TCR as described herein associated (usually by fusion to an N- or C-terminus of the alpha or beta chain) with an anti-CD3 antibody, or a functional fragment or variant of said anti-CD3 antibody. Antibody fragments and variants/analogues which are suitable for use in the compositions and methods described herein include minibodies, Fab fragments, F(ab<′>)2fragments, dsFv and scFv fragments, Nanobodies™ (Ablynx (Belgium), molecules comprising synthetic single immunoglobulin variable heavy chain domain derived from a camelid (e.g. camel or llama) antibody) and Domain Antibodies (comprising an affinity matured single immunoglobulin variable heavy chain domain or immunoglobulin variable light chain domain (Domantis (Belgium)) or alternative protein scaffolds that exhibit antibody-like binding characteristics such as Affibodies (comprising engineered protein A scaffold Affibody (Sweden)) or Anticalins (comprising engineered anticalins Pieris (German)).

The therapeutic agent may preferably be selected from the group consisting of an immune effector molecule, a cytotoxic agent and a radionuclide. Preferably, the immune effector molecule is a cytokine.

The pharmacokinetic modifying moiety may be for example at least one polyethylene glycol repeating unit, at least one glycol group, at least one sialyl group or a combination thereof. The association of at least one polyethylene glycol repeating unit, at least one glycol group, at least one sialyl group may be caused in a number of ways known to those skilled in the art. In a preferred embodiment the units are covalently linked to the TCR. The TCRs according to the invention can be modified by one or several pharmacokinetic modifying moieties. In particular, the soluble form of the TCR is modified by one or several pharmacokinetic modifying moieties. The pharmacokinetic modifying moiety may achieve beneficial changes to the pharmacokinetic profile of the therapeutic, for example improved plasma half-life, reduced or enhanced immunogenicity, and improved solubility.

The TCR according to the invention may be soluble or membrane bound. The term “soluble” refers to a TCR being in soluble form (i.e. having no transmembrane or cytoplasmic domains), for example for use as a targeting agent for delivering therapeutic agents to the antigen presenting cell. For stability, soluble αβ heterodimeric TCRs preferably have an introduced disulfide bond between residues of the respective constant domains, as described, for example, in WO 03/020763. One or both of the constant domains present in an αβ heterodimer of the invention may be truncated at the C terminus or C termini, for example by up to 15, or up to 10 or up to 8 or fewer amino acids. For use in adoptive therapy, an αβ heterodimeric TCR may, for example, be transfected as full length chains having both cytoplasmic and transmembrane domains. TCRs may contain a disulfide bond corresponding to that found in nature between the respective alpha and beta constant domains, additionally or alternatively a non-native disulfide bond may be present.

The TCR, in particular a soluble form of the TCR according to the invention, can thus be modified by attaching additional functional moieties, e.g. for reducing immunogenicity, increasing hydrodynamic size (size in solution) solubility and/or stability (e.g. by enhanced protection to proteolytic degradation) and/or extending serum half-life.

Other useful functional moieties and modifications include “suicide” or “safety switches” that can be used to shut off effector host cells carrying an inventive TCR in a patient's body. An example is the inducible Caspase 9 (iCasp9) “safety switch” described by Gargett and Brown Front Pharmacol. 2014; 5: 235. Briefly, effector host cells are modified by well-known methods to express a Caspase 9 domain whose dimerization depends on a small molecule dimerizer drug such as AP1903/CIP, and results in rapid induction of apoptosis in the modified effector cells. The system is for instance described in EP2173869 (A2). Examples for other “suicide” “safety switches” are known in the art, e.g. Herpes Simplex Virus thymidine kinase (HSV-TK), expression of CD20 and subsequent depletion using anti-CD20 antibody or myc tags (Kieback et al, Proc Natl Acad Sci USA. 2008 Jan. 15; 105(2):623-8).

TCRs with an altered glycosylation pattern are also envisaged herein. As is known in the art, glycosylation patterns can depend on the amino acid sequence (e.g., the presence or absence of particular glycosylation amino acid residues, discussed below) and/or the host cell or organism in which the protein is produced. Glycosylation of polypeptides is typically either N-linked or O-linked. N-linked refers to the attachment of the carbohydrate moiety to the side chain of an asparagine residue. Addition of N-linked glycosylation sites to the binding molecule is conveniently accomplished by altering the amino acid sequence such that it contains one or more tri-peptide sequences selected from asparagine-X-serine and asparagine-X-threonine (where X is any amino acid except proline). O-linked glycosylation sites may be introduced by the addition of or substitution by, one or more serine or threonine residues to the starting sequence.

Another means of glycosylation of TCRs is by chemical or enzymatic coupling of glycosides to the protein. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. Similarly, deglycosylation (i.e., removal of carbohydrate moieties present on the binding molecule) may be accomplished chemically, e.g. by exposing the TCRs to trifluoromethanesulfonic acid, or enzymatically by employing endo- and exo-glycosidases.

It is also conceivable to add a drug such as a small molecule compound to the TCR, in particular a soluble form of the inventive TCR. Linkage can be achieved via covalent bonds, or non-covalent interactions such as through electrostatic forces. Various linkers, known in the art, can be employed in order to form the drug conjugates.

The TCR, in particular a soluble form of the inventive TCR can additionally be modified to introduce additional domains which aid in identification, tracking, purification and/or isolation of the respective molecule (tags). Thus in some embodiments, the TCR α chain or the TCR β chain may be modified to comprise an epitope tag.

Epitope tags are useful examples of tags that can be incorporated into the TCR of the invention. Epitope tags are short stretches of amino acids that allow for binding of a specific antibody and therefore enable identification and tracking of the binding and movement of soluble TCRs or host cells within the patient's body or cultivated (host) cells. Detection of the epitope tag, and hence, the tagged TCR, can be achieved using a number of different techniques.

Tags can further be employed for stimulation and expansion of host cells carrying an inventive TCR by cultivating the cells in the presence of binding molecules (antibodies) specific for said tag.

In general, the TCR can be modified in some instances with various mutations that modify the affinity and the off-rate of the TCR with the target antigen. In particular, the mutations may increase the affinity and/or reduce the off-rate. Thus, the TCR may be mutated in at least one CDR and the variable domain framework region thereof.

However, in a preferred embodiment the CDR regions of the TCR are not modified or in vitro affinity maturated such as for the TCR receptors in the examples. This means that the CDR regions have naturally occurring sequences. This can be advantageous, since in vitro affinity maturation may lead to immunogenicity to the TCR molecule. This may lead to the production of anti-drug antibodies decreasing or inactivating the therapeutic effect and the treatment and/or induce adverse effects.

The mutation may be one or more substitution(s), deletion(s) or insertions(s). These mutations may be introduced by any suitable method known in the art, such as polymerase chain reaction, restriction enzyme based cloning, ligation independent cloning procedures, which are described for Example in Sambrook, Molecular Cloning—4^{th} Edition (2012) Cold Spring Harbor Laboratory Press.

Theoretically, unpredictable TCR specificity with the risk for cross-reactivity can occur due to mispairing between endogenous and exogenous TCR chains. To avoid mispairing of TCR sequences, the recombinant TCR sequence may be modified to contain minimal murinized Ca and CP regions, a technology that has been shown to efficiently enhance correct pairing of several different transduced TCR chains. Murinization of TCRs (i.e. exchanging the human constant regions in the alpha and beta chain by their murine counterparts) is a technique that is commonly applied in order to improve cell surface expression of TCRs in host cells. Without wishing to be bound by specific theory, it is thought that murinized TCRs associate more effectively with CD3 co-receptors; and/or that preferentially pair with each other and are less prone to form mixed TCRs on human T cells genetically modified ex vivo to express the TCRs of desired antigenic specificity, but still retaining and expressing their “original” TCRs.

Nine amino acids responsible for the improved expression of murinized TCRs have been identified (Sommermeyer and Uckert, J Immunol. 2010 Jun. 1; 184(11):6223-31) and it is envisaged to substitute one or all of the amino acid residues in the TCRs alpha and/or beta chain constant region for their murine counterpart residues. This technique is also referred to as “minimal murinization”, and offers the advantage of enhancing cell surface expression while, at the same time, reducing the number of “foreign” amino acid residues in the amino acid sequence and, thereby, the risk of immunogenicity.

Some embodiments refer to an isolated TCR as described herein, wherein the TCR is of the single chain type, wherein the TCR α chain and the TCR β chain are linked by a linker sequence.

A suitable single chain TCR form comprises a first segment constituted by an amino acid sequence corresponding to a variable TCR α region, a second segment constituted by an amino acid sequence corresponding to a variable TCR β region fused to the N terminus of an amino acid sequence corresponding to a TCR β chain constant region extracellular sequence, and a linker sequence linking the C terminus of the first segment to the N terminus of the second segment. Alternatively, the first segment may be constituted by an amino acid sequence corresponding to a TCR β chain variable region, the second segment may be constituted by an amino acid sequence corresponding to a TCR α chain variable region sequence fused to the N terminus of an amino acid sequence corresponding to a TCR α chain constant region extracellular sequence. The above single chain TCRs may further comprise a disulfide bond between the first and second chains, and wherein the length of the linker sequence and the position of the disulfide bond being such that the variable domain sequences of the first and second segments are mutually orientated substantially as in native T cell receptors. More specifically the first segment may be constituted by an amino acid sequence corresponding to a TCR α chain variable region sequence fused to the N terminus of an amino acid sequence corresponding to a TCR α chain constant region extracellular sequence, the second segment may be constituted by an amino acid sequence corresponding to a TCR β chain variable region fused to the N terminus of an amino acid sequence corresponding to TCR β chain constant region extracellular sequence, and a disulfide bond may be provided between the first and second chains. The linker sequence may be any sequence which does not impair the function of the TCR.

In the context of the present invention, a “functional” TCR α and/or β chain fusion protein shall mean a TCR or TCR variant, for example modified by addition, deletion or substitution of amino acids, that maintains at least substantial biological activity. In the case of the α and/or β chain of a TCR, this shall mean that both chains remain able to form a T-cell receptor (either with a non-modified α and/or β chain or with another inventive fusion protein α and/or β chain) which exerts its biological function, in particular binding to the specific peptide-MHC complex of said TCR, and/or functional signal transduction upon specific peptide:MHC interaction.

In specific embodiments the TCR may be modified, to be a functional T-cell receptor (TCR) α and/or β chain fusion protein, wherein said epitope-tag has a length of between 6 to 15 amino acids, preferably 9 to 11 amino acids. In another embodiment the TCR may be modified to be a functional T-cell receptor (TCR) α and/or β chain fusion protein wherein said T-cell receptor (TCR) α and/or β chain fusion protein comprises two or more epitope-tags, either spaced apart or directly in tandem. Embodiments of the fusion protein can contain 2, 3, 4, 5 or even more epitope-tags, as long as the fusion protein maintains its biological activity/activities (“functional”).

Preferred is a functional T-cell receptor (TCR) α and/or β chain fusion protein according to the present invention, wherein said epitope-tag is selected from, but not limited to, CD20 or Her2/neu tags, or other conventional tags such as a myc-tag, FLAG-tag, T7-tag, HA (hemagglutinin)-tag, His-tag, S-tag, GST-tag, or GFP-tag. myc, T7, GST, GFP tags are epitopes derived from existing molecules. In contrast, FLAG is a synthetic epitope tag designed for high antigenicity (see, e.g., U.S. Pat. Nos. 4,703,004 and 4,851,341). The myc tag can preferably be used because high quality reagents are available to be used for its detection. Epitope tags can of course have one or more additional functions, beyond recognition by an antibody. The sequences of these tags are described in the literature and well known to the person of skill in art.

### TCR Variants

Another aspect of the invention refers to a polypeptide comprising a functional portion of the TCR of as described herein, wherein the functional portion comprises at least one of the amino acid sequences of SEQ ID NOs: 4 and 7.

The functional portion may mediate the binding of the TCR to the antigen, in particular to the antigen-MHC complex.

In one embodiment, the functional portion comprises the TCR α variable chain and/or the TCR variable chain as described herein.

The TCR variant molecule may have the binding properties of the TCR receptor but may be combined with signaling domains of effectors cells (other than T cells), in particular with signaling domains of NK cells. Therefore, some embodiments refer to a protein comprising a functional portion of the TCR as described herein in combination with the signaling domains of an effector cell, such as a NK cell.

Another aspect of the invention refers to a multivalent TCR complex comprising at least two TCRs as described herein. In one embodiment of this aspect, at least two TCR molecules are linked via linker moieties to form multivalent complexes. Preferably, the complexes are water soluble, so the linker moiety should be selected accordingly. It is preferable that the linker moiety is capable of attaching to defined positions on the TCR molecules, so that the structural diversity of the complexes formed is minimized. One embodiment of the present aspect is provided by a TCR complex of the invention wherein the polymer chain or peptidic linker sequence extends between amino acid residues of each TCR which are not located in a variable region sequence of the TCR. Since the complexes of the invention may be for use in medicine, the linker moieties should be chosen with due regard to their pharmaceutical suitability, for example their immunogenicity. Examples of linker moieties which fulfil the above desirable criteria are known in the art, for example the art of linking antibody fragments.

Examples for linkers are hydrophilic polymers and peptide linkers. An example for hydrophilic polymers are polyalkylene glycols. The most commonly used of this class are based on polyethylene glycol or PEG. However, others are based on other suitable, optionally substituted, polyalkylene glycols which include polypropylene glycol, and copolymers of ethylene glycol and propylene glycol. Peptide linkers are comprised of chains of amino acids, and function to produce simple linkers or multimerization domains onto which TCR molecules can be attached.

One embodiment refers to a multivalent TCR complex, wherein at least one of said TCRs is associated with a therapeutic agent.

### Cytokine and Chemokine Release

Some embodiments refer to the isolated TCR as described herein, polypeptide as described herein, multivalent TCR complex as described herein, wherein IFN-γ secretion is induced by binding of the inventive TCR expressed on an effector cell to the HLA-A*02 bound form of the amino acid sequence selected from the group consisting of SEQ ID NO: 1.

The IFN-γ secretion induced by binding of the inventive TCR expressed on an effector cell to the HLA-A*02 bound form of the amino acid sequence selected from the group consisting of SEQ ID NO:1 may be more than 500 pg/ml, more preferably more than 1000 pg/ml, most preferably more than 2000 pg/ml. The IFN-γ secretion may be at least 5 times higher when binding to the HLA-A*02 bound form of the amino acid sequence of SEQ ID NO: 1 compared to binding to the HLA-A*02 bound form of an irrelevant peptide (e.g. SEQ ID No: 22).

The “effector cell” may be a peripheral blood lymphocyte (PBL) or a peripheral blood mononuclear cell (PBMC). Typically, the effector cell is an immune effector cell, especially a T cell. Other suitable cell types include gamma-delta T cells and NK-like T cells.

The invention relates also to methods for identifying a TCR or a fragment thereof that binds to the target amino acid sequence SEQ ID NO: 1 or the HLA-A*02, preferably or the HLA-A*02:01 bound form thereof, wherein the method comprises contacting the candidate TCR or antigen binding fragment thereof with the amino acid sequences of SEQ ID NO: 1 or the HLA-A*02, preferably or the HLA-A*02:01 bound form thereof and determining whether the candidate TCR or antigen binding fragment thereof binds to the target and/or mediates an immune response. Whether the candidate TCR or antigen binding fragment thereof mediates an immune response can be determined for example by the measurement of cytokine secretion, such as IFN-γ secretion. As described above cytokine secretion may be measured by an in vitro assay in which K562 cells, transduced with HLA-A2 (or other APCs transduced with HLA-A2) transfected with ivtRNA coding the amino acid sequence SEQ ID NO: 1 are incubated with CD8+ enriched PBMC expressing the TCR or a molecule comprising a fragment of the TCR to be investigated.

The TCR of the invention is particularly useful, since it shows high tumor cell recognition capacity. Further the TCR as described herein exhibits high tumor cell killing capacity. In addition, the TCR of the invention has a high functional avidity.

Moreover, the TCR shows favourable cytokine release pattern, which is advantageous for effective tumor regression.

### Nucleic Acids, Vectors

Another aspect of the invention refers to a nucleic acid encoding a TCR as described herein or encoding the polynucleotide encoding a TCR as described herein.

| Peptide | nucleotide | |
|---|---|---|
| sequence | sequence | |
| SEQ ID NO | SEQ ID NO | description |
| 2 | 12 | TCR α chain CDR1 |
| 3 | 13 | TCR α chain CDR2 |
| 4 | 14 | TCR α chain CDR3 |
| 5 | 15 | TCR β chain CDR1 |
| 6 | 16 | TCR β chain CDR2 |
| 7 | 17 | TCR β chain CDR3 |
| 8 | 18 | TCR α chain variable region |
| 9 | 19 | TCR β chain variable region |
| 10 | 20 | TCR α chain complete (murinized constant |
| | | region) |
| 11 | 21 | TCR β chain complete (murinized constant |
| | | region) |

“Nucleic acid molecule” and generally means a polymer of DNA or RNA, which can be single-stranded or double-stranded, synthesized or obtained (e.g., isolated and/or purified) from natural sources which can contain natural, non-natural or altered nucleotides, and which can contain a natural, non-natural or altered internucleotide linkage, such as a phosphoroamidate linkage or a phosphorothioate linkage instead of the phosphodiester found between the nucleotides of an unmodified oligonucleotide. Preferably, the nucleic acids described herein are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication. The nucleic acids can be constructed based on chemical synthesis and/or enzymatic ligation reactions using procedures known in the art or commercially available (e.g. from Genscript, Thermo Fisher and similar companies). See, for example, Sambrook et al. for example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). The nucleic acid can comprise any nucleotide sequence which encodes any of the recombinant TCRs, polypeptides, or proteins, or functional portions or functional variants thereof.

The present disclosure also provides variants of the isolated or purified nucleic acids wherein the variant nucleic acids comprise a nucleotide sequence that has at least 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence encoding the TCR described herein. Such variant nucleotide sequence encodes a functional TCR that specifically recognizes MAGEA10.

The disclosure also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the TCRs described herein. It is generally appreciated that conditions can be rendered more stringent by the addition of increasing amounts of formamide.

As already described elsewhere herein, the nucleic acid encoding the TCR may be modified. Useful modifications in the overall nucleic acid sequence may be codon optimization. Alterations may be made which lead to conservative substitutions within the expressed amino acid sequence. These variations can be made in complementarity determining and non-complementarity determining regions of the amino acid sequence of the TCR chain that do not affect function. Usually, additions and deletions should not be performed in the CDR3 region.

Another embodiment refers to a vector comprising the nucleic acid encoding the TCR as described herein.

The vector is preferably a plasmid, shuttle vector, phagemide, cosmid, expression vector, retroviral vector, adenoviral vector or particle and/or vector to be used in gene therapy.

A “vector” is any molecule or composition that has the ability to carry a nucleic acid sequence into a suitable host cell where synthesis of the encoded polypeptide can take place. Typically, and preferably, a vector is a nucleic acid that has been engineered, using recombinant DNA techniques that are known in the art, to incorporate a desired nucleic acid sequence (e.g. a nucleic acid of the invention). The vector may comprise DNA or RNA and/or comprise liposomes. The vector may be a plasmid, shuttle vector, phagemide, cosmid, expression vector, retroviral vector, lentiviral vector, adenoviral vector or particle and/or vector to be used in gene therapy. A vector may include nucleic acid sequences that permit it to replicate in a host cell, such as an origin of replication. A vector may also include one or more selectable marker genes and other genetic elements known to those of ordinary skill in the art. A vector preferably is an expression vector that includes a nucleic acid according to the present invention operably linked to sequences allowing for the expression of said nucleic acid.

Preferably, the vector is an expression vector. More preferably, the vector is a retroviral, more specifically a gamma-retroviral or lentiviral vector.

### Cells, Cell Lines

Another aspect of the invention refers to a cell expressing the TCR as described herein.

In some embodiments, the cell is isolated or non-naturally occurring.

In specific embodiments, the cell may comprise the nucleic acid encoding the TCR as described herein or the vector comprising said nucleic acid.

In the cell the above described vector comprising a nucleic acid sequence coding for the above described TCR may be introduced or ivtRNA coding for said TCR may be introduced. The cell may be a peripheral blood lymphocyte such as a T cell. The method of cloning and exogenous expression of the TCR is for example described in Engels et al. (Relapse or eradication of cancer is predicted by peptide-major histocompatibility complex affinity. Cancer Cell, 23(4), 516-26. 2013). The transduction of primary human T cells with a lentiviral vector is, for example, described in Cribbs “simplified production and concentration of lentiviral vectors to achieve high transduction in primary human T cells” BMC Biotechnol. 2013; 13: 98.

The term “transfection” and “transduction” are interchangeable and refer to the process by which an exogenous nucleic acid sequence is introduced in a host cell, e.g. in an eukaryotic host cell. It is noted that introduction or transfer of nucleic acid sequences is not limited to the mentioned methods but can be achieved by any number of means including electroporation, microinjection, gene gun delivery, lipofection, superfection and the mentioned infection by retroviruses or other suitable viruses for transduction or transfection.

Some embodiments refer to a cell comprising:

a) an expression vector which comprises at least one nucleic acid as described herein, or

b) a first expression vector which comprises a nucleic acid encoding the alpha chain of the TCR as described herein, and a second expression vector which comprises a nucleic acid encoding the beta chain of a TCR as described herein.

In some embodiments, the cell is a peripheral blood lymphocyte (PBL) or a peripheral blood mononuclear cell (PBMC). The cell may be a natural killer cell or a T cell. Preferably, the cell is a T cell. The T cell may be a CD4+ or a CD8+ T cell. In some embodiments the cell is a stem cell like memory T cell.

Stem cell-like memory T cells (TSCM) are a less-differentiated subpopulation of CD8+ or CD4+ T cells, which are characterized by the capacity of self-renewal and to persist long-term. Once these cells encounter their antigen in vivo, they differentiate further into central memory T cells (TCM), effector memory T cells (TEM) and terminally differentiated effector memory T cells (TEMRA) with some TSCM remaining quiescent (Flynn et al., Clinical & Translational Immunology (2014). These remaining TSCM cells show the capacity to build a durable immunological memory in vivo and therefore are considered an important T cell subpopulation for adoptive T cell therapy (Lugli et al., Nature Protocols 8, 33-42 (2013) Gattinoni et al., Nat. Med. 2011 October; 17(10): 1290-1297). Immune-magnetic selection can be used in order to restrict the T cell pool to the stem cell memory T cell subtype see (Riddell et al. 2014, Cancer Journal 20(2): 141-44)

### Antibodies Targeting TCR

Another aspect of the invention refers to an antibody or antigen binding fragment thereof specifically binding to a portion of the TCR as described herein that mediates specificity for MAGEA10. In one embodiment, the portion of the TCR that mediates the MAGEA10 specificity comprises the CDR3 of the alpha chain of SEQ ID NO: 4 and/or the CDR3 of the beta chain of SEQ ID NO: 7.

The antibody antigen binding fragment may modulate the activity of the TCR. It may block or may not block the binding of the TCR with MAGEA10. It could be used for modulating the therapeutic activity of the TCR or for diagnostic purposes.

### Pharmaceutical Compositions, Medical Treatments and Kits

Another aspect of the invention refers to pharmaceutical composition comprising the TCR as described herein, the polypeptide comprising a functional portion of said TCR, the multivalent TCR complex as described herein, the nucleic acid encoding the TCR, the vector comprising said nucleic acid, the cell comprising said TCR, or the antibody specifically binding to a portion of the TCR as described herein.

Those active components of the present invention are preferably used in such a pharmaceutical composition, in doses mixed with an acceptable carrier or carrier material, that the disease can be treated or at least alleviated. Such a composition can (in addition to the active component and the carrier) include filling material, salts, buffer, stabilizers, solubilizers and other materials, which are known state of the art.

The term “pharmaceutically acceptable” defines a non-toxic material, which does not interfere with effectiveness of the biological activity of the active component. The choice of the carrier is dependent on the application.

The pharmaceutical composition may contain additional components which enhance the activity of the active component or which supplement the treatment. Such additional components and/or factors can be part of the pharmaceutical composition to achieve synergistic effects or to minimize adverse or unwanted effects.

Techniques for the formulation or preparation and application/medication of active components of the present invention are published in “Remington's Pharmaceutical Sciences”, Mack Publishing Co., Easton, Pa., latest edition. An appropriate application is a parenteral application, for example intramuscular, subcutaneous, intramedular injections as well as intrathecal, direct intraventricular, intravenous, intranodal, intraperitoneal or intratumoral injections. The intravenous injection is the preferred treatment of a patient.

According to a preferred embodiment, the pharmaceutical composition is an infusion or an injection.

An injectable composition is a pharmaceutically acceptable fluid composition comprising at least one active ingredient, e.g. an expanded T cell population (for example autologous or allogenic to the patient to be treated) expressing a TCR. The active ingredient is usually dissolved or suspended in a physiologically acceptable carrier, and the composition can additionally comprise minor amounts of one or more non-toxic auxiliary substances, such as emulsifying agents, preservatives, and pH buffering agents and the like. Such injectable compositions that are useful for use with the fusion proteins of this disclosure are conventional; appropriate formulations are well known to those of ordinary skill in the art.

Typically, the pharmaceutical composition comprises at least one pharmaceutically acceptable carrier.

Accordingly, another aspect of the invention refers to the TCR as described herein, the polypeptide comprising a functional portion of said TCR, the multivalent TCR complex according as described herein, the nucleic acid encoding said TCR, the vector comprising said nucleic acid, the cell comprising said TCR, or the antibody specifically binding to a portion of the TCR as described herein for use as a medicament.

Some embodiments refer to the TCR as described herein, the polypeptide comprising a functional portion of said TCR, the multivalent TCR complex according as described herein, the nucleic acid encoding said TCR, the vector comprising said nucleic acid, the cell comprising said TCR for use in the treatment of cancer.

In one embodiment the cancer is a hematological cancer or a solid tumor.

Hematological cancers also called blood cancers which do not form solid tumors and therefore are dispersed in the body. Examples of hematological cancers are leukemia, lymphoma or multiple myeloma. There are two major types of solid tumors, sarcomas and carcinomas. Sarcomas are for example tumors of the blood vessel, bone, fat tissue, ligament, lymph vessel, muscle or tendon.

In one embodiment, the cancer is selected from the group consisting of prostate cancer, uterine cancer, thyroid cancer, testicular cancer, renal cancer, pancreatic cancer, ovarian cancer, esophageal cancer, non-small-cell lung cancer, lung adenocarcinoma, squamous cell carcinoma, non-Hodgkin's lymphoma, multiple myeloma, melanoma, hepatocellular carcinoma, head and neck cancer, gastric cancer, endometrial cancer, cervical cancer, colorectal cancer, stomach adenocarcinoma, cholangiocarcinoma, breast cancer, bladder cancer, myeloid leukemia and acute lymphoblastic leukemia, sarcoma or osteosarcoma.

Also contemplated herein are pharmaceutical compositions and kits containing one or more of (i) an isolated TCR as described herein; (ii) viral particles comprising a nucleic acid encoding a recombinant TCR; (iii) immune cells, such as T cells or NK cells, modified to express a recombinant TCR as described herein; (iv) nucleic acids encoding a recombinant TCR as described herein. In some embodiments, the present disclosure provides compositions comprising lentiviral vector particles comprising a nucleotide sequence encoding a recombinant TCR described herein (or T cells that have been modified using the vector particles described herein to express a recombinant TCR). Such compositions can be administered to subjects in the methods of the present disclosure as described further herein.

Compositions comprising the modified T cells as described herein can be utilized in methods and compositions for adoptive immunotherapy in accordance with known techniques, or variations thereof that will be apparent to those skilled in the art based on the instant disclosure.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

The number of cells for an effective treatment in the composition is typically greater than 10 cells, and up to 10⁶, up to and including 10⁸ or 10⁹ cells and can be more than 10¹⁰ cells. The number of cells will depend upon the ultimate use for which the composition is intended as will the type of cells included therein. For uses provided herein, the cells are generally in a volume of a liter or less, can be 500 ml or less, even 250 ml or 100 ml or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁹, 10¹⁰ or 10¹¹ cells. Pharmaceutical compositions provided herein can be in various forms, e.g., in solid, liquid, powder, aqueous, or lyophilized form. Examples of suitable pharmaceutical carriers are known in the art. Such carriers and/or additives can be formulated by conventional methods and can be administered to the subject at a suitable dose. Stabilizing agents such as lipids, nuclease inhibitors, polymers, and chelating agents can preserve the compositions from degradation within the body. In a composition intended to be administered by injection, one or more of a surfactant, preservative, wetting agent, dispersing agent, suspending agent, buffer, stabilizer and isotonic agent may be included.

The recombinant TCRs as described herein, or the viral vector particles comprising a nucleotide sequence encoding a recombinant TCR provided herein, can be packaged as kits. Kits can optionally include one or more components such as instructions for use, devices, and additional reagents, and components, such as tubes, containers and syringes for practice of the methods. Exemplary kits can include the nucleic acids encoding the recombinant TCRs, the recombinant TCR polypeptides, or viruses provided herein, and can optionally include instructions for use, a device for detecting a virus in a subject, a device for administering the compositions to a subject, and a device for administering the compositions to a subject.

Kits comprising polynucleotides encoding a gene of interest (e.g., a recombinant TCR) are also contemplated herein. Kits comprising a viral vector encoding a sequence of interest (e.g., a recombinant TCR) and optionally, a polynucleotide sequence encoding an immune checkpoint inhibitor are also contemplated herein.

Kits contemplated herein also include kits for carrying out the methods for detecting the presence of polynucleotides encoding any one or more of the TCRs disclosed herein. In particular, such diagnostic kits may include sets of appropriate amplification and detection primers and other associated reagents for performing deep sequencing to detect the polynucleotides encoding TCRs disclosed herein. In further embodiments, the kits herein may comprise reagents for detecting the TCRs disclosed herein, such as antibodies or other binding molecules. Diagnostic kits may also contain instructions for determining the presence of the polynucleotides encoding the TCRs disclosed herein or for determining the presence of the TCRs disclosed herein. A kit may also contain instructions. Instructions typically include a tangible expression describing the components included in the kit, and methods for administration, including methods for determining the proper state of the subject, the proper dosage amount, and the proper administration method. Instructions can also include guidance for monitoring the subject over the duration of the treatment time.

Kits provided herein also can include a device for administering a composition described herein to a subject. Any of a variety of devices known in the art for administering medications or vaccines can be included in the kits provided herein. Exemplary devices include, but are not limited to, a hypodermic needle, an intravenous needle, a catheter, a needle-less injection device, an inhaler, and a liquid dispenser, such as an eyedropper. Typically, the device for administering a virus of the kit will be compatible with the virus of the kit; for example, a needle-less injection device such as a high pressure injection device can be included in kits with viruses not damaged by high pressure injection, but is typically not included in kits with viruses damaged by high pressure injection.

Kits provided herein also can include a device for administering a compound, such as a T cell activator or stimulator, or a TLR agonist, such as a TLR4 agonist to a subject. Any of a variety of devices known in the art for administering medications to a subject can be included in the kits provided herein. Exemplary devices include a hypodermic needle, an intravenous needle, a catheter, a needle-less injection, but are not limited to, a hypodermic needle, an intravenous needle, a catheter, a needle-less injection device, an inhaler, and a liquid dispenser such as an eyedropper. Typically, the device for administering the compound of the kit will be compatible with the desired method of administration of the compound.

### EXPERIMENTS

### Example 1: MAGE-A10^{SLL}-TCR-Transgenic T Cells Bind MAGE-A10^{SLL}-Multimer

An in vitro priming approach to isolate MAGE-A10^{SLL}-reactive T-cell clones was used. The priming system used mature dendritic cells (mDCs) of an HLA-A*02:01 positive donor as antigen-presenting cells and autologous CD8⁺-enriched T cells as responding cells. In vitro transcribed RNA (ivtRNA) encoding 31 amino acids of the human MAGE A10 gene (PRAHAEIRKMSLLKFLAKVNGSDPRSFPL) served as the source of specific antigen. After electroporation into the mDCs, the MAGE-A10-encoding ivtRNA was translated into protein, which was subsequently processed and presented as peptides by HLA-A*02:01 molecules on the mDCs. In vitro co-cultures of T cells with the ivtRNA-transfected mDCs from the same donor led to de novo induction of antigen-specific T cells. Antigen-specific T cells were enriched by FACS-based single cell sorting. Sequences of TCR alpha and TCR beta chains of MAGE-A10^{SLL}-reactive T-cell clones were identified by Next Generation Sequencing. Both constant TCR regions were replaced by their murine counterparts. The nucleotide sequence of the murinized TCR was codon optimized using the GeneOptimizer™ algorithm (ThermoFisher) and cloned into the retroviral vector pES.12-6. PBMCs of a healthy donor were isolated by ficoll gradient centrifugation. CD8 T-cells were enriched by negative magnetic selection (Miltenyi) and stimulated in non-tissue culture 24-well plates with anti-CD3 and anti-CD28 mAb (BD Pharmingen, Heidelberg, Germany). Amphotropic retroviral particles were produced by transfection of HEK293T cells with the respective TCR encoding retroviral plasmid and two expression plasmids. On day two after stimulation, CD8 T cells were transduced and on day twelve enriched for transduced CD8⁺ cells by FACS using the murine constant beta region as a marker for transduction and then expanded by the rapid expansion protocol (Riddell S R, Science, 1992 Jul. 10; 257(5067):238-41).

### Results:

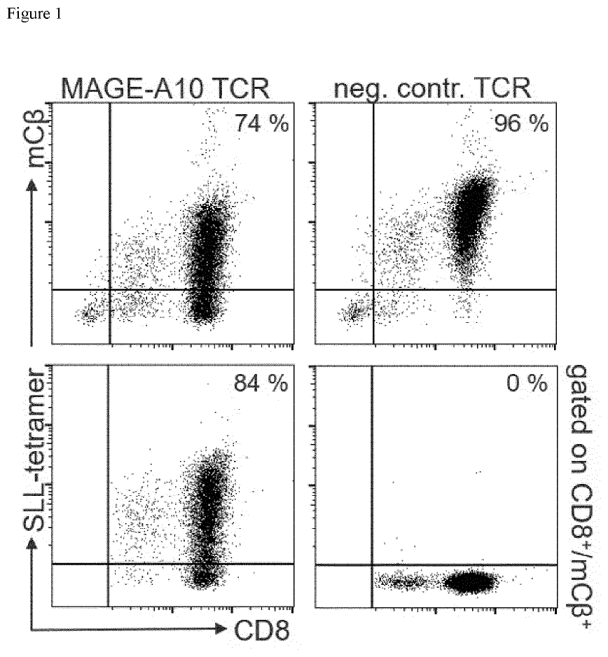

CD8 T cells were transduced with a TCR isolated from a MAGE-A10^{SLL}-reactive T-cell clone and a control TCR that did not recognize MAGE-A10. They were stained with a MAGE-A10^{SLL}-MHC-multimer and antibodies against CD8 and the murine constant beta region. The TCR beta chain was expressed on 74% of the CD8 T cells for the MAGE-A10^{SLL}-TCR and 96% for the negative control TCR. The majority of the MAGE-A10^{SLL}-TCR transduced cells (84%) bound the MAGE-A10^{SLL}-MHC-multimer. No MAGE-A10^{SLL}-MHC-multimer-staining was observed with the negative control TCR. These results show that TCRs isolated from MAGE-A10^{SLL}-reactive T-cell clones can be transgenically expressed in T cells of a healthy donor (FIG. 1).

### Example 2: MAGE-A10^{SLL}-TCR-Transgenic T Cells Recognize the MAGE-A10^{SLL}-Peptide

To assure functionality of the transgenically expressed, recombinant TCR, TCR-transduced effector T cells were co-cultured with peptide-loaded T2 cells (target cells). T2 cells loaded with 10⁻⁵M of the MAGE-A10-derived SLL peptide (SEQ ID NO: 1) served as positive target, T2 cells loaded with an irrelevant peptide (SEQ ID NO: 22) served as negative target. As effector cells, CD8-enriched T cells were transduced to stably express the recombinant MAGE-A10^{SLL}-TCR or the negative control TCR (TCR specific for the non-relevant peptide according to SEQ ID NO: 22). Effector cells and target cells were co-cultured in an E:T of 2:1 in 96-well round-bottom plates. After ˜20 h of co-culture, the supernatants were harvested and analyzed by standard sandwich ELISA.

### Results:

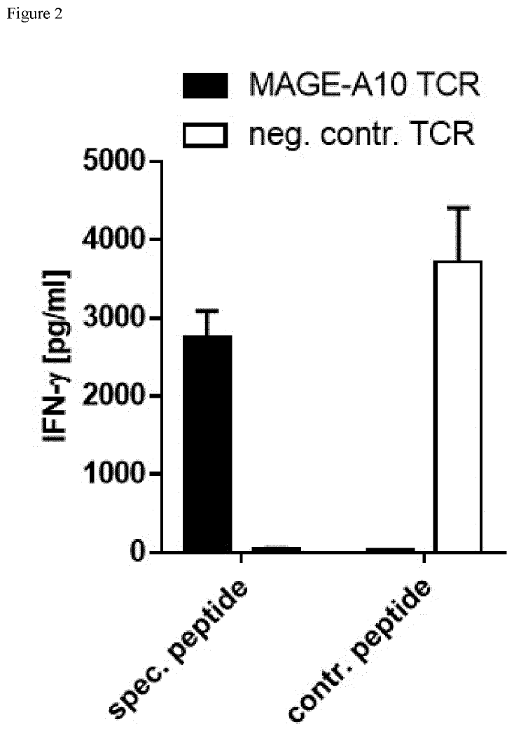

MAGE-A10^{SLL}-TCR-expressing effector cells only secreted IFN-γ upon cultivation with MAGE-A10^{SLL}-peptide-loaded T2 cells, indicating a specific recognition of the presented peptide:MHC complex. The negative target, T2 cells loaded with the control peptide, were only recognized by the negative control TCR (FIG. 2).

### Example 3: MAGE-A10^{SLL}-TCR-Transgenic T Cells Recognize and Kill HLA-A*02:01/MAGE-A10-Positive Tumor Cell Lines

To analyze the TCR's efficacy, specificity and its suitability for clinical application, a set of tumor cell lines was tested for TCR-mediated recognition by measuring IFN-γ secretion and cytotoxicity upon co-culture with TCR-transduced effector cells.

For the IFN-γ release assay, effector cells were transduced with the MAGE-A10^{SLL}-TCR or the negative control TCR and co-cultured with different tumor cell lines. Cell lines A375, NCI-H1755 and UACC-62 (HLA-A2+/MAGEA10+) and HLA-A2-transfected cell line KYO-1 (HLA-A2+/MAGEA10+) served as positive-targets. Cell lines Mel624.38 and 647-V (HLA-A2+/MAGEA10−) served as negative targets. HLA-A2-expression of the cell lines was analyzed by flow cytometry and MAGEA10-expression data were generated using qPCR or extracted from the publicly available databases known to the person skilled in the art (data not shown). Effector and target cells were seeded in 96-well round-bottom plates at an E:T of 2:1, supernatants were harvested after ˜20 h of co-culture and levels of IFN-γ were analyzed by standard sandwich ELISA. For the cytotoxicity assay, an IncuCyte™ Zoom device was used to perform a long-term killing assay over three days. The IncuCyte™ device is a microscope-based system that allows live imaging of cells. A set of tumor cell lines (HLA-A2+/MAGEA10+: A375, NCI-H1755 and UACC-62, HLA-A2+/MAGEA10−: Mel624.38 and 647-V) was seeded into the wells of a 96-well flat-bottom plate (20,000 cells per well) and co-cultured with TCR-expressing effector T cells (100,000 cell per well). The tumor cell lines were stably expressing the nuclear restricted red fluorescent protein mKate2, enabling the IncuCyte™ Zoom System to determine the amount of red fluorescent cells in each well (Total Integrated Intensity in RCU×μm²/Image, RCU=red calibration unit) at multiple time points over a total time period of 72 hours. An increased cell number per well over time would signify an outgrowth of the tumor cells, while a reduction of the cell number per well would indicate the TCR-mediated cytotoxicity. of fluorescent target cells.

### Results:

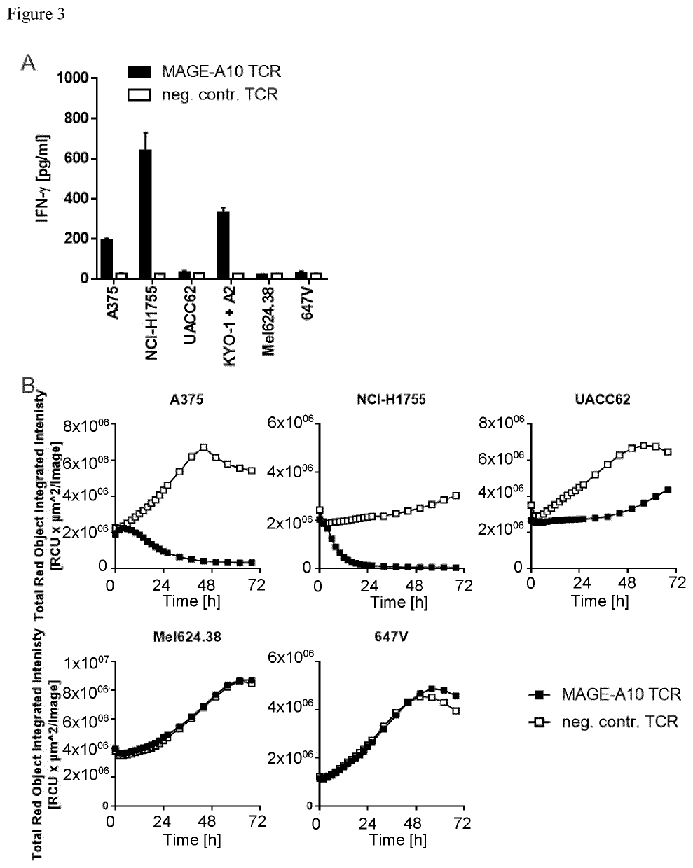

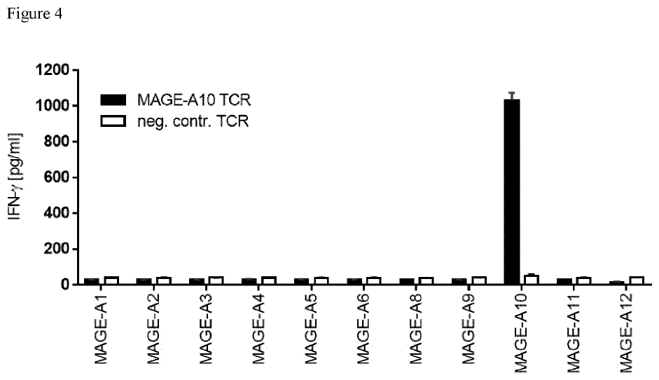

The MAGE-A10^{SLL}-TCR-transgenic T cells released IFN-γ when co-cultured with the HLA-A2+/MAGEA10+ tumor cell lines A375, NCI-H1755 and KYO-1 but not in response to the HLA-A2+/MAGEA10− cell lines Mel624.38 and 647-V. Also, the A2+/MAGEA10+ tumor cell line UACC62 was not recognized. T cells expressing the negative control TCR did not recognize any of the tumor cell lines. The HLA-A2+/MAGEA10+ tumor cell lines A375 and NCI-H1755 were killed by MAGE-A10-TCR-transgenic T cells and the growth of UACC62 cells was inhibited. HLA-A2+/MAGEA10− cell lines Mel624.38 and 647-V were not killed and negative control TCR expressing T cells did not lyze any of the tumor cell lines. These results show that MAGE-A10^{SLL}-TCR-transgenic T cells can recognize MAGE-A10^{SLL}-peptide on HLA-A*02 at physiological levels on tumor cell lines and that they can mediate cytotoxicity against these cells (FIGS. 3A and 4B).

### Example 4: Analysis of Potential Cross-Recognition of Other MAGE-A Family Members

As other members of the MAGE-A family can contain peptide sequences highly similar to the MAGE-A10^{SLL}-peptide, a potential TCR-mediated cross-recognition of other MAGE-A family members was further investigated. The expression of MAGE-A family members is described not only in cancers and testis, but also in vital organs, qualifying a potential cross-recognition of protein-derived peptides to be an exclusion criterion for a TCR for clinical development.

To investigate the recognition of endogenously processed and presented peptides originating from other MAGE-A family members, they were recombinantly expressed in HLA-A2-positive K562 cells. By co-culturing cell lines, expressing the recombinant MAGE-A family members with MAGE-A10^{SLL}-TCR-expressing effector cells, the potential cross-recognition of peptides derived from MAGE family members was analyzed.

HLA-A2⁺ K562 cells were transduced with constructs encoding the following MAGE family members: MAGE-A1 (NCBI Reference Sequence (accession): NM_004988.5), MAGE-A2 (NM_005361.3), MAGE-A3 (NM_005362.4), MAGE-A4 (NM_001011548.1), MAGE-A5 (NM_021049.4), MAGE-A6 (NM_005363.3), MAGE-A8 (NM_001166400.1), MAGE-A9 (NM_005365.4), MAGE-A11 (NM_005366.4), MAGE-A12 (NM_001166386.3) and co-cultured with MAGE-A10^{SLL}-TCR or the negative control TCR expressing T cells. Supernatants were harvested after ˜20 h of co-culture and secreted IFN-γ was analyzed by standard sandwich ELISA.

### Results:

MAGE-A10^{SLL}-TCR expressing T cells only recognized MAGE-A10 positive HLA-A2⁺ K562 cells but not HLA-A2⁺ K562 cells transduced with any other MAGE-A family member (FIG. 4).

### Example 5: HLA-Fine-Typing for the MAGE-A10^{SLL}-TCR and the Analysis of Potential Alloreactivity

As TCRs recognize their specific peptide only in combination with a certain MHC molecule, not only the level of antigen-expression but also the HLA-type of a target cell defines a target to be a positive or negative target. Dependent on the HLA-type, a patient can or cannot be included into a treatment regimen for TCR-based ACT.

To evaluate, which HLA-molecules, in addition to HLA-A*02:01, can present the MAGEA10-derived SLL peptide and can be recognized by MAGE-A10^{SLL}-expressing T cells, a detailed restriction analysis was performed. Therefore, 53 lymphoblastoid cell lines (LCL), covering the most frequent HLA-allotypes in Europeans and North American-Caucasians were used as antigen presenting cells. They were used unloaded or loaded with the SLL peptide (10⁻⁵ M; SEQ ID NO 1) for the co-culture with TCR-expressing effector cells. The co-culture was setup with an E:T of 2:1 in 96-well round-bottom plates. Supernatants were harvested after ˜20 h and levels of IFN-γ were analyzed by standard sandwich ELISA.

### Results:

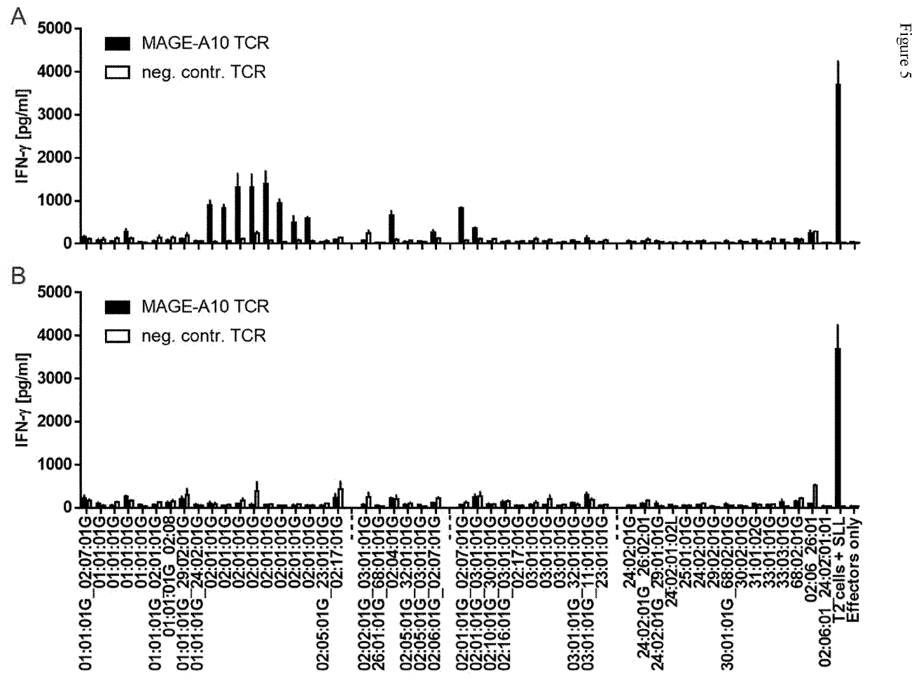

The restriction analysis for the MAGE-A10^{SLL}-TCR indicates the TCR's potential to recognize the MAGEA10-derived epitope not only presented on HLA-A*02:01 but also on HLA-A*02:04. MAGE-A10^{SLL}-TCR expressing T cells showed no cross-recognition of any HLA, as unloaded LCL were not recognized (FIGS. 5A and 5B).

### Example 6: Analysis of Potential Recognition of Normal Cells

To investigate a TCR's safety profile, the TCR-mediated recognition of cells derived from healthy tissues of different origin has to be investigated and excluded. Therefore, TCR-expressing effectors were co-cultured with normal cells derived from the kidney (renal cortical epithelial cells, HRCEpC), lung (lung fibroblasts, NHLF), and iPS-derived hepatocytes, cardiomyocytes, astrocytes and endothelial cells (EC). The investigated cells were endogenously negative for the antigen MAGE-A10 and therefore allowed the identification of potential MAGE-A10-unrelated off-target toxicities. The normal cells were thawed and cultivated for one week prior the co-culture as specified by the suppliers. The cells were seeded in 96-well flat-bottom plates and co-cultured with MAGE-A10^{SLL}-TCR or the negative control TCR expressing T cells. HLA-A2-expression of the target cells was confirmed by antibody-staining and flow cytometry (data not shown). Target cells were either tested unmodified or loaded with excessive amounts (10⁻⁵M) of the MAGE-A10^{SLL}-peptide as a positive control. After ˜20 h of co-culture, supernatants were harvested and levels of IFN-γ were analyzed by standard sandwich ELISA.

### Results:

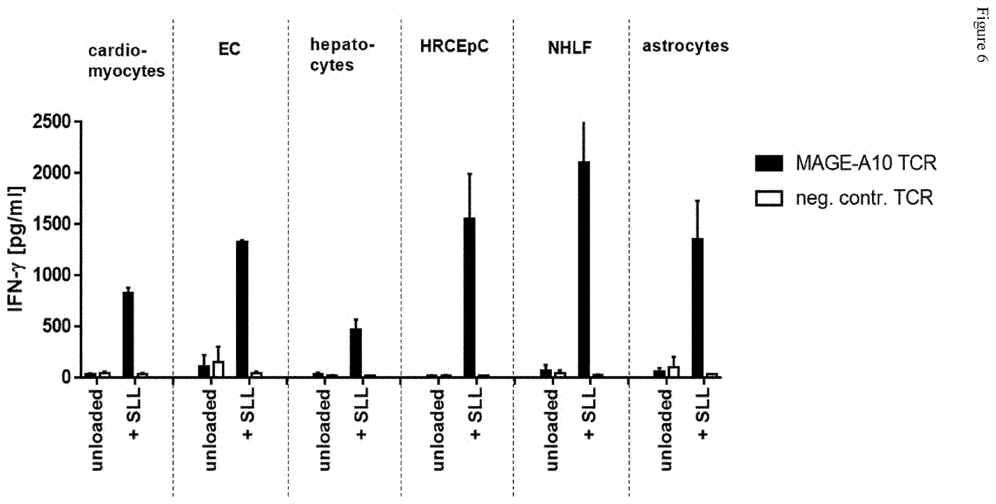

MAGE-A10^{SLL}-TCR expressing T cells released IFN-γ in response to all MAGE-A10^{SLL}-peptide-loaded cells, representing the target cells potential to properly present TCR epitopes on their cell surface. The unmodified normal cells were not recognized by effectors expressing the transgenic MAGE-A10^{SLL}-TCR, indicating a favorable safety profile of the investigated TCR (FIG. 6).

The application further comprises the following items:

Item 1: Isolated T cell receptor (TCR) specific for MAGEA10.

Item 2: Isolated TCR according to item 1, wherein the TCR specifically recognizes the amino acid sequence SEQ ID NO: 1 or a fragment thereof.

Item 3: Isolated TCR according to items 1 and 2, wherein the TCR specifically recognizes the HLA-A2 bound form of the amino acid sequence of SEQ ID NO: 1.

Item 4: Isolated TCR according to any of the preceding items, wherein the TCR specifically recognizes the amino acid sequence of SEQ ID NO: 1, which is presented by a molecule encoded by a gene selected from the group consisting of HLA-A*02:01 and HLA-A*02:04.

Item 5:Isolated TCR according to any of the preceding items, wherein the TCR specifically recognizes the amino acid sequence of SEQ ID NO: 1, which is presented by the HLA-A*02:01 encoded molecule.

Item 6: Isolated TCR according to any one of the preceding items, wherein the TCR comprises
a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4,
a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7.

Item 7: Isolated TCR according to any one of the preceding items, wherein the TCR comprises a variable TCR α region having an amino acid sequence which is at least 80% identical to SEQ ID NO: 8 and a variable TCR β region having an amino acid sequence which is at least 80% identical to SEQ ID NO: 9.

Item 8: Isolated TCR according to any one of the preceding embodiments, wherein the TCR comprises

a variable TCR α region having the amino acid sequence of SEQ ID NO: 8 and a variable TCR β region having the amino acid sequence of SEQ ID NO: 9.

Item 9: Isolated TCR according to any one of the preceding items, wherein the TCR comprises a TCR α chain having an amino acid sequence which is at least 80% identical to SEQ ID NO: 10 and a TCR β chain having an amino acid sequence which is at least 80% identical to SEQ ID NO: 11.

Item 10: Isolated TCR according to any one of the preceding items, wherein the TCR comprises

a TCR α chain having the amino acid sequence of SEQ ID NO: 8 or SEQ ID NO: 10 and a TCR chain having the amino acid sequence of SEQ ID NO: 9 or SEQ ID NO: 11.

Item 11: Isolated TCR according to any one of the preceding items, wherein the TCR comprises a TCR α chain and a TCR β chain, wherein
the variable TCR α region has an amino acid sequence which is at least 80% identical to SEQ ID NO: 8 and comprises a CDR3 having the amino acid sequence set out in SEQ ID NO: 4,
the variable TCR β region has an amino acid sequence which is at least 80% identical to SEQ ID NO: 9 and comprises a CDR3 having the amino acid sequence set out in SEQ ID NO: 7.

Item 12: Isolated TCR according to any one of the preceding items, wherein the TCR is purified.

Item 13: Isolated TCR according to any one of the preceding items, wherein its amino acid sequence comprises one or more phenotypically silent substitutions.

Item 14: Isolated TCR according to any one of the preceding items, wherein its amino acid sequence is modified to comprise a detectable label, a therapeutic agent or pharmacokinetic modifying moiety.

Item 15: Isolated TCR according to item 14, wherein the therapeutic agent is selected from the group consisting of an immune effector molecule, a cytotoxic agent and a radionuclide.

Item 16: Isolated TCR according to item 15, wherein the immune effector molecule is a cytokine.

Item 17: Isolated TCR according to any one of the preceding items, wherein the TCR is soluble or membrane bound.

Item 18: Isolated TCR according to item 14, wherein the pharmacokinetic modifying moiety is at least one polyethylene glycol repeating unit, at least one glycol group, at least one sialyl group or a combination thereof.

Item 19: Isolated TCR according to any one of the preceding items, wherein the TCR is of the single chain type, wherein the TCR α chain and the TCR β chain are linked by a linker sequence.

Item 20: Isolated TCR according to items 1 to 19, wherein the TCR α chain or the TCR β chain is modified to comprise an epitope tag.

Item 21: Isolated polypeptide comprising a functional portion of the TCR of any of items 1 to 20, wherein the functional portion comprises at least one of the amino acid sequences of SEQ ID NOs: 4 and 7.

Item 22: Isolated polypeptide comprising a functional portion of the TCR of any of items 1 to 20, wherein the functional portion comprises the amino acid sequences of SEQ ID NOs: 2, 3, 4, 5, 6, and 7.

Item 23: Isolated polypeptide according to item 21, wherein the functional portion comprises the TCR α variable chain and/or the TCR β variable chain.

Item 24: Multivalent TCR complex comprising the TCR as embodied in any one of items 1 to 20.

Item 25: Isolated TCR according to items 1 to 20, polypeptide according to items 21 to 23, multivalent TCR complex according to item 24, wherein IFN-γ secretion is induced by binding to the amino acid sequence of SEQ ID NO: 1, which is presented by the HLA-A*02:01 encoded molecule.

Item 26: Nucleic acid encoding a TCR according to any one of items 1 to 20 or encoding the polypeptide according to items 21 to 23.

Item 27: Vector comprising the nucleic acid of item 26.

Item 28: Vector according to item 27, wherein the vector is an expression vector.

Item 29: Vector according to item 27 or 28, wherein the vector is a retroviral vector.

Item 30: Vector according to item 27 or 28, wherein the vector is a lentiviral vector.

Item 31: Cell expressing the TCR according to items 1 to 20.

Item 32: Cell according to item 31, wherein the cell is isolated or non-naturally occurring.

Item 33: Cell according to items 31 and 32, wherein the cell comprises the nucleic acid according to item 26 or the vector according to items 27 to 30.

Item 34: Cell according to items 31 to 33, wherein the cell comprises:

a) an expression vector which comprises at least one nucleic acid as embodied in item 26, or

b) a first expression vector which comprises a nucleic acid encoding the alpha chain of the TCR as embodied in any one of the items 1 to 20, and a second expression vector which comprises a nucleic acid encoding the beta chain of a TCR as embodied in any one of the items 1 to 20.

Item 35: Cell according to any one of items 31 to 34, wherein the cell is a peripheral blood lymphocyte (PBL) or a peripheral blood mononuclear cell (PBMC).

Item 36: Cell according to any one of items 31 to 35, wherein the cell is a T cell.

Item 37: Antibody or antigen binding fragment thereof specifically binding to a portion of the TCR according to items 1 to 20 that mediates specificity for MAGEA10.

Item 38: Antibody according to item 37, wherein the portion of the TCR that mediates the MAGEA10 specificity comprises at least one of the CDRs set out in SEQ ID NOs: 2, 3, 4, 5, 6, and 7, preferably the CDR3 of the alpha chain of SEQ ID NO: 4 and/or the CDR3 of the beta chain of SEQ ID NO: 7, more preferably the CDRs of the alpha chain set out in SEQ ID NOs: 2, 3 and 4, and the CDRs of the beta chain of SEQ ID 5, 6 and 7.

Item 39: Pharmaceutical composition comprising the TCR according to items 1 to 20, the polypeptide according to items 21 to 23, the multivalent TCR complex according to item 24 the nucleic acid according to item 26, the vector according to items 27 to 30, the cell according to any one of items 31 to 36, or the antibody according to items 37 to 38.

Item 40: Pharmaceutical composition according to item 39 wherein the pharmaceutical composition comprises at least one pharmaceutically acceptable carrier.

Item 41: The TCR according to items 1 to 20, the polypeptide according to items 21 to 23, the multivalent TCR complex according to item 24, the nucleic acid according to item 26, the vector according to items 27 to 30, the cell according to any one of items 31 to 36, or the antibody according to items 37 to 38 for use as a medicament.

Item 42: The TCR according to items 1 to 20, the polypeptide according to items 21 to 23, the multivalent TCR complex according item 24, the nucleic acid according to item 26, the vector according to claims 27 to 30 or the cell according to any one of items 31 to 36 for use in the treatment of cancer.

Item 43: The TCR, the polypeptide, the multivalent TCR complex, the nucleic acid, the vector or the cell according to item 42, wherein the cancer is a hematological cancer or a solid tumor.

Item 44: The TCR, the polypeptide, the multivalent TCR complex, the nucleic acid, the vector or the cell according to items 42 and 43, wherein the cancer is selected from the group consisting of prostate cancer, uterine cancer, thyroid cancer, testicular cancer, renal cancer, pancreatic cancer, ovarian cancer, esophageal cancer, non-small-cell lung cancer, lung adenocarcinoma, squamous cell carcinoma, non-Hodgkin's lymphoma, multiple myeloma, melanoma, hepatocellular carcinoma, head and neck cancer, gastric cancer, endometrial cancer, cervical cancer, colorectal cancer, stomach adenocarcinoma, cholangiocarcinoma, breast cancer, bladder cancer, myeloid leukemia and acute lymphoblastic leukemia, sarcoma or osteosarcoma.

Item 45: The TCR, the polypeptide, the multivalent TCR complex, the nucleic acid, the vector or the cell according to items 42 and 43, wherein the cancer is preferably selected from the group consisting sarcoma or osteosarcoma.

1. Isolated T cell receptor (TCR) specific for MAGEA10.

2. Isolated TCR according to claim 1, wherein the TCR specifically recognizes the amino acid sequence SEQ ID NO: 1 or a fragment thereof.

3. Isolated TCR according to claims 1 and 2, wherein the TCR specifically recognizes the HLA-A2 bound form of the amino acid sequence of SEQ ID NO: 1.

4. Isolated TCR according to any of the preceding claims, wherein the TCR specifically recognizes the amino acid sequence of SEQ ID NO: 1, which is presented by a molecule encoded by a gene selected from the group consisting of HLA-A*02:01 and HLA-A*02:04, preferably wherein the TCR specifically recognizes the amino acid sequence of SEQ ID NO: 1, which is presented by the HLA-A*02:01 encoded molecule.

5. Isolated TCR according to any one of the preceding claims, wherein the TCR comprises
a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4,
a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7.

6. Isolated TCR according to any one of the preceding claims, wherein the TCR comprises a variable TCR α region having an amino acid sequence which is at least 80% identical to SEQ ID NO: 8 and a variable TCR β region having an amino acid sequence which is at least 80% identical to SEQ ID NO: 9.

7. Isolated polypeptide comprising a functional portion of the TCR of any of claims 1 to 6, wherein the functional portion comprises at least one of the amino acid sequences of SEQ ID NOs: 4 and 7, preferably wherein the functional portion comprises the amino acid sequences of SEQ ID NOs: 2, 3, 4, 5, 6, and 7.

8. Multivalent TCR complex comprising a least two TCRs as claimed in any one of claims 1 to 6.

9. Isolated TCR according to claims 1 to 6, polypeptide according to claim 7, multivalent TCR complex according to claim 8, wherein IFN-γ secretion is induced by binding to the amino acid sequence of SEQ ID NO: 1, which is presented by the HLA-A*02:01 encoded molecule.

10. Nucleic acid encoding a TCR according to any one of claims 1 to 6 or encoding the isolated polypeptide according to claim 7.

11. Vector comprising the nucleic acid of claim 10, wherein the vector is preferably an expression vector, more preferably a retroviral vector or lentiviral vector.

12. Cell expressing the TCR according to claims 1 to 6.

13. Antibody or antigen binding fragment thereof specifically binding to a portion of the TCR according to claims 1 to 6 that mediates specificity for MAGEA10, preferably wherein the portion of the TCR that mediates the MAGEA10 specificity comprises the CDR3 of the alpha chain of SEQ ID NO: 4 and/or the CDR3 of the beta chain of SEQ ID NO: 7.

14. The TCR according to claims 1 to 6, the polypeptide according to claim 7, the multivalent TCR complex according to claim 8, the nucleic acid according to claim 10, the vector according to claim 11, the cell according to claim 12, or the antibody according to claim 13 for use as a medicament.

15. The TCR according to claims 1 to 6, the polypeptide according to claim 7, the multivalent TCR complex according to claim 8, the nucleic acid according to claim 10, the vector according to claim 11, the cell according to claim 12, or the antibody according to claim 13 for use in the treatment of cancer, wherein the cancer is selected from the group consisting of prostate cancer, uterine cancer, thyroid cancer, testicular cancer, renal cancer, pancreatic cancer, ovarian cancer, esophageal cancer, non-small-cell lung cancer, lung adenocarcinoma, squamous cell carcinoma, non-Hodgkin's lymphoma, multiple myeloma, melanoma, hepatocellular carcinoma, head and neck cancer, gastric cancer, endometrial cancer, cervical cancer, colorectal cancer, stomach adenocarcinoma, cholangiocarcinoma, breast cancer, bladder cancer, myeloid leukemia and acute lymphoblastic leukemia, sarcoma or osteosarcoma.


SEQUENCE LISTING (23 sequences)

SEQ ID NO: 1 - protein, 9 aa, Homo sapiens
SLLKFLAKV

SEQ ID NO: 2 - protein, 6 aa, Homo sapiens
NSMFDY

SEQ ID NO: 3 - protein, 7 aa, Homo sapiens
ISSIKDK

SEQ ID NO: 4 - protein, 13 aa, Homo sapiens
CAANSNSGYALNF

SEQ ID NO: 5 - protein, 5 aa, Homo sapiens
KGHDR

SEQ ID NO: 6 - protein, 6 aa, Homo sapiens
SFDVKD

SEQ ID NO: 7 - protein, 13 aa, Homo sapiens
CATGDPLQETQYF

SEQ ID NO: 8 - protein, 139 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAANSNSGYALNFGKGTSLLVTPH

SEQ ID NO: 9 - protein, 131 aa, Homo sapiens
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATGDPLQETQYFGPGTRLLVL

SEQ ID NO: 10 - protein, 275 aa, Artificial Sequence
  with murinized constant region
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAANSNSGYALNFGKGTSLLVTPHIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 11 - protein, 304 aa, Artificial Sequence
  with murinized constant region
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATGDPLQETQYFGPGTRLLVLEDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 12 - DNA, 18 bp, Homo sapiens
aactctatgttcgactac

SEQ ID NO: 13 - DNA, 21 bp, Homo sapiens
atcagcagcatcaaggacaag

SEQ ID NO: 14 - DNA, 39 bp, Homo sapiens
tgcgccgccaatagcaatagcggctacgccctgaatttc

SEQ ID NO: 15 - DNA, 15 bp, Homo sapiens
aagggccacgaccgg

SEQ ID NO: 16 - DNA, 18 bp, Homo sapiens
agcttcgacgtgaaggac

SEQ ID NO: 17 - DNA, 39 bp, Homo sapiens
tgcgccactggcgaccctctgcaagagacacagtatttc

SEQ ID NO: 18 - DNA, 415 bp, Homo sapiens
atggctatgctgctgggagcctctgtgctgatcctgtggctgcaacccgattgggtcaacagccagcagaagaacgacgaccagcaagtgaagcagaacagccccagcctgagcgtgcaagagggcagaatcagcatcctgaactgcgactacaccaactctatgttcgactacttcctgtggtacaagaagtaccccgccgagggaccgaccttcctgatcagcatcagcagcatcaaggacaagaacgaggacggccggttcaccgtgtttctgaacaagagcgccaagcacctgagcctgcacatcgtgccttctcagcctggcgatagcgccgtgtatttctgcgccgccaatagcaatagcggctacgccctgaatttcggcaagggcacaagcctgctggtcacccctc

SEQ ID NO: 19 - DNA, 394 bp, Homo sapiens
atggccagcctgctgttcttctgcggcgccttttatctgctcggcaccggctctatggacgccgacgttacacagacccctcggaacagaatcaccaagaccggcaagcggatcatgctggaatgcagccagaccaagggccacgaccggatgtactggtacagacaggatccaggactgggcctgagactgatctactacagcttcgacgtgaaggacatcaacaagggcgagatcagcgacggctacagcgtgtcaagacaggcccaggccaagttcagcctgagcctggaaagcgctatccccaaccagacagccctgtacttctgcgccactggcgaccctctgcaagagacacagtatttcggccctggcaccagactgctggttctgg

SEQ ID NO: 20 - DNA, 825 bp, Artificial Sequence
  with murinized constant region; codon optimized
atggctatgctgctgggagcctctgtgctgatcctgtggctgcaacccgattgggtcaacagccagcagaagaacgacgaccagcaagtgaagcagaacagccccagcctgagcgtgcaagagggcagaatcagcatcctgaactgcgactacaccaactctatgttcgactacttcctgtggtacaagaagtaccccgccgagggaccgaccttcctgatcagcatcagcagcatcaaggacaagaacgaggacggccggttcaccgtgtttctgaacaagagcgccaagcacctgagcctgcacatcgtgccttctcagcctggcgatagcgccgtgtatttctgcgccgccaatagcaatagcggctacgccctgaatttcggcaagggcacaagcctgctggtcacccctcacatccagaatccggagcccgccgtataccagctgaaggaccctagaagccaggacagcaccctgtgcctgttcaccgacttcgacagccagatcaacgtgcccaagaccatggaaagcggcaccttcatcaccgacaagacagtgctggacatgaaggccatggacagcaagtccaacggcgcaatcgcctggtccaaccagaccagcttcacatgccaggacatcttcaaagagacaaacgccacataccccagcagcgacgtgccctgtgatgccaccctgacagagaagtccttcgagacagacatgaacctgaacttccagaatctgtccgtgatgggcctgagaatcctgctgctgaaggtggccggcttcaatctgctgatgaccctgcggctgtggtccagc

SEQ ID NO: 21 - DNA, 912 bp, Artificial Sequence
  with murinized constant region; codon optimized
atggccagcctgctgttcttctgcggcgccttttatctgctcggcaccggctctatggacgccgacgttacacagacccctcggaacagaatcaccaagaccggcaagcggatcatgctggaatgcagccagaccaagggccacgaccggatgtactggtacagacaggatccaggactgggcctgagactgatctactacagcttcgacgtgaaggacatcaacaagggcgagatcagcgacggctacagcgtgtcaagacaggcccaggccaagttcagcctgagcctggaaagcgctatccccaaccagacagccctgtacttctgcgccactggcgaccctctgcaagagacacagtatttcggccctggcaccagactgctggttctggaagatctccggaacgtgaccccccctaaagtgaccctgttcgaacccagcaaggccgagatcgccaacaagcagaaagccaccctcgtgtgcctggccagaggcttcttccccgaccatgtggaactgtcttggtgggtcaacggcaaagaggtgcacagcggagtgtccaccgaccctcaggcctacaaagagagcaactacagctactgcctgagcagcagactgcgggtgtccgccaccttctggcacaacccccggaaccacttcagatgccaggtgcagtttcacggcctgagcgaagaggacaagtggcccgaaggctcccccaagcccgtgacccagaatatctctgccgaggcctggggcagagccgactgtggaattaccagcgccagctaccaccagggcgtgctgtctgccaccatcctgtacgagatcctgctgggcaaggccaccctgtacgccgtgctggtgtctggcctggtgctgatggccatggtcaagaagaagaacagc

SEQ ID NO: 22 - protein, 10 aa, Homo sapiens
KLYGLDWAEL

SEQ ID NO: 23 - protein, 29 aa, Homo sapiens
PRAHAEIRKMSLLKFLAKVNGSDPRSFPL